(19) United States
(12) Patent Application Publication
(10) Pub. No.: US 2014/0083437 A1
(43) Pub. Date: Mar. 27, 2014

(54) ARM BOARD DEVICE

(71) Applicants: Eliessa Florence Hall, St. Augustine, FL (US); William Hall, St. Augustine, FL (US)

(72) Inventors: Eliessa Florence Hall, St. Augustine, FL (US); William Hall, St. Augustine, FL (US)

(21) Appl. No.: 14/004,407

(22) PCT Filed: Mar. 14, 2012
(86) PCT No.: PCT/US12/29043

§ 371 Date: Nov. 25, 2013

Related U.S. Application Data
(60) Provisional application No. 61/452,569, filed on Mar. 14, 2011.
(60) Provisional application No. 61/560,926, filed on Nov. 17, 2011.

(51) Int. Cl. A61F 5/37; A61B 5/022

(52) U.S. Cl. CPC A61F 5/373; A61B 5/022
USPC 128/879

(57) ABSTRACT

The disclosure relates to an arm board device to replace the need for physical arm restraints. An exemplary arm board device comprises an arm support and, optionally, a hand support.

### CROSS REFERENCE TO RELATED APPLICATIONS

This application claims priority to and incorporates by reference the disclosures of U.S. Provisional Patent Application No. 61/452,569 entitled “B.O.A.R.D.—BILATERAL ORTHOPEDIC ANTI-RESTRANT DEVICE—A BENT ARM BOARD”, filed on Mar. 14, 2011 and U.S. Provisional Patent Application No. 61/560,926 entitled “ARM BOARD DEVICE”, filed on Nov. 17, 2011.

### BACKGROUND OF THE INVENTION

1. Field of the Invention

The disclosure relates to an arm board device to replace the need for physical arm restraints.

2. Discussion of the Related Art

Various medical devices (e.g., endotracheal tubes, nasogastric/oral gastric tubes, tracheostomy tubes, arterial lines, monitoring equipment, Foley catheters, PICC lines, IVs, etc.) may be used by medical practitioners in the care of their patients. Whether due to a lack of mental alertness or orientation (e.g., because of age, a medical condition, sedative medication, and/or anesthesia), or even due to defiance, patients may inadvertently or intentionally attempt to remove such medical devices (e.g., self-extubation). Such attempts, and if successful, such removal, can in turn lead to increased medical complications, injury, and even death.

To prevent such removal, current approaches comprise physical wrist restraints whereby straps tied to a hospital bed are secured around a patient's wrists, thus severely limiting or immobilizing the patient's arms and hands. This current practice has limited effectiveness immobilizing the patient's arms. Additionally, many problems are associated with the use of physical restraints, including increased length of stay in the medical care setting; increased incidence of falls and other injuries; increased nosocomial infections and pressure ulcers; increased risk of complications from immobility of the extremity including brachial plexus injury, joint contractures, muscle weakness, pressure ulcers, agitation, delirium, pneumonia, psychological distress, restlessness; and increased risk of mortality.

Patient and family members report feeling as though current restraint usage is overly restrictive, cruel and hurtful to the patient. Further complicating this problem is that attempts to remove physical wrist restraints can lead to increased medical complications such as removal of invasive monitoring equipment and airway support devices such as endotracheal tubes. Like attempts to remove medical devices, a patient's attempts to remove restraints can similarly lead to further injury, longer stays in the medical care setting, and even death. In addition to potential medical and physical complications, restraint use can also lead to significant psychological complications for the patient and emotional strain for the patient's family.

The primary focus of medical support is to maximize healing, restore function, and prevent further injury for all critically ill medical, surgical, injured, and neurologically impaired patients. In an acute state of injury, patients are unable to protect themselves from further injury and compromise. Having the tools necessary to provide a safe, healing environment, while minimizing any potential negative consequences or complications, is a fundamental goal for each medical practitioner.

One restraint-related device in the prior art covers a large portion of the patient's arm, thus negating the ability for the medical practitioner to monitor or use this portion of the patient's arm for intravenous access. In addition, the device offers no support for the patient's hand and is a straight-arm splint, thus offering only a limited comfort level.

Another related device in the prior art does not provide any support for the patient's hand or elbow, and similarly comprises only a straight-arm splint. The device does not prevent the removal of invasive lines and medical devices, and does not provide support for the hand or arm to maximize patient comfort and safety.

Yet another related device in the prior art has limited applicability for the critical care patient due to its channel design and the depth at which the patient's arm rests within the device. Furthermore, the device is not an effective alternative to restraints because of its short length and, therefore, its inability to provide a structure that would support the patient's arm sufficiently to prevent flexion thereof. In addition, the device does not allow for support of the patient's hand for preventing dislodgement of invasive equipment and medical devices.

Thus, there exists a need in the art for alternatives to physical arm restraints.

### SUMMARY OF THE INVENTION

According to one aspect of the disclosure, an arm board device prevents a patient from removing various medical devices used by medical practitioners in the care of the patient. In various embodiments, an arm board device provides for freedom of arm movement at the shoulder (e.g., abduction and adduction of the arm) as well as hand movement. An exemplary arm board device is not classified as a restraint by regulatory agencies such as the Centers for Medicare Services (C.M.S.) and the Joint Commission on Accreditation of Healthcare Organizations (Joint Commission). According to the Conditions of Participation with Seclusion (C.O.P.S.) study (2009), any IV arm board, unless tied down or attached to the bed, is not considered a restraint.

According to another aspect of the disclosure, an arm board device provides added safety over current approaches, including decreased patient injury due to falling out of bed and decreased incidence of skin breakdown in hands, forearms, or elbows. The arm board device may also provide for decreased incidence of deep vein thrombosis (“DVT”) in upper extremities.

According to yet another aspect of the disclosure, an arm board device reduces other significant medical, physical, and psychological complications for the patient and emotional strain for the patient's family.

### BRIEF DESCRIPTION OF THE DRAWINGS

The accompanying drawings are included to provide a further understanding of the disclosure and are incorporated in and constitute a part of this specification, illustrate embodiments of the disclosure, and together with the description serve to explain the principles of the disclosure, wherein:

FIG. 1 illustrates a front view of an exemplary left arm board device;

FIG. 2A illustrates a front view of an arm support of an exemplary arm board device;

FIG. 2B illustrates a back view of an arm support of an exemplary arm board device;

FIG. 2C illustrates a back view of a distal end of an arm support of an exemplary arm board device;

FIGS. 3A-3B illustrate left hand front and right hand back views, respectively, of a hand support of an exemplary arm board device.

### DETAILED DESCRIPTION OF THE ILLUSTRATED EMBODIMENTS

Persons skilled in the art will readily appreciate that various aspects of the present disclosure may be realized by any number of methods and apparatuses configured to perform the intended functions. Stated differently, other methods and apparatuses may be incorporated herein to perform the intended functions. It should also be noted that the accompanying drawing figures referred to herein are not all drawn to scale, but may be exaggerated to illustrate various aspects of the present disclosure, and in that regard, the drawing figures should not be construed as limiting. Finally, although the present disclosure may be described in connection with various principles and beliefs, the present disclosure should not be bound by theory.

“Proximal” as used herein means a portion of a device that, in use, is located closer to the patient's heart. “Distal” as used herein means a portion of a device that, in use, is located further from the patient's heart. According to one aspect of the disclosure, an arm board device prevents a patient from removing various medical devices used by medical practitioners in the care of patients. These devices include, for example, endotracheal tubes, nasogastric/oral gastric tubes, tracheostomy tubes, arterial lines, monitoring equipment, and Foley catheters, to name several. These types of medical devices, and the device disclosed herein, may be used by medical practitioners in the care of the patient in, for example, intensive care settings, rehabilitation centers, nursing homes, dentistry offices, and the like. In various embodiments, an arm board device provides for freedom of arm movement at the shoulder (e.g., abduction and adduction of the arm) and hand movement.

According to another aspect of the disclosure, an arm board device provides added safety over current approaches, including, for example, decreased patient injury from falling out of bed and decreased incidence of skin breakdown in hands, forearms, or elbows. The arm board device may also provide for decreased incidence of DVT in upper extremities.

According to yet another aspect of the disclosure, an arm board device reduces other significant medical, physical, and psychological complications for the patient and emotional strain for the patient's family.

An exemplary arm board device comprises an arm support and, optionally, a hand support.

The arm support is generally configured to secure the arm such that an obtuse angle is maintained between the patient's upper and lower arm limbs. In various embodiments, the angle is maintained at from about 125 to about 165 degrees, or more preferably, at about 145 degrees. The arm support can be further configured to allow for from about 10 to about 30 degrees of freedom, for example, with a hinge that may or may not be adjusted, to facilitate performance of various active daily living (“ADL”) tasks. The arm support can be configured to conform to the anterior, or more preferably, the posterior (i.e., dorsal) side of the arm. In this regard, the arm support can comprise a curve orthogonal to a longitudinal axis of the arm support to provide a configuration suitable for receiving a patient's arm that can extend along all or a portion of the arm support. In various embodiments, the curved or contoured configuration is not so deep as to cover portions of the patient's upper or lower arm limbs that need to be accessed by various medical devices. The arm support may be configured to extend distally from and/or proximally to the elbow any suitable distance. In various embodiments, the distal and/or proximal extension beyond the elbow is not so far as to cover portions of the patient's upper or lower arm limbs that need to be accessed by various medical devices, including, for example, sphygmomanometer cuffs. Structural support can be added to the arm support by providing one or more structural support elements such as, for example, a C-channel. In various embodiments, the arm support comprises a C-channel configured along all or a portion of the length of each longitudinal edge of the back surface of the arm support. In other embodiments, C-channels on the edges of the arm support may be combined with other structural support, for example, gussets located elsewhere on the arm support. In these embodiments, the C-channels are not limited to a “C”-shaped configuration or any particular cross-section, but may comprise channels having a “C”-shaped cross section, a “U”-shaped cross section, or any variety of channel section generally known or yet to be discovered. In accordance with the embodiments, and as described in more detail below, the C-channel may also provide for slidable engagement or coupling of a hand support component.

Structural support elements may begin at a distal end of the arm support and taper down to, or end before, a proximal end of the arm support. This exemplary embodiment may reduce interference by the arm support with medical monitoring devices and, for example, permit improved accuracy of readings from sphygmomanometer cuffs. In various embodiments, the structural support extends uniformly along the full length of the longitudinal edges of the arm support and the arm support is symmetrical along both a longitudinal axis and a transverse axis. In other embodiments, the arm support is asymmetrical and structural support elements extend along the full length of the longitudinal axis, allowing the same arm support to be used for either a right patient arm or a left patient arm, with the appropriate hand support being attachable to the arm support at opposite ends based on the arm to which the device is applied. A proximal end of the arm support may further be curved dorsally at the proximal end to reduce any skin breakdown in arms. In various embodiments, the arm support is symmetrical about at least one longitudinal axis.

The optional hand support is generally configured to support the hand such that the angle between the patient's hand and lower arm limb is maintained at from about 160 to about 200 degrees, or more preferably, at about 180 degrees (i.e., flat). In various embodiments, the hand support comprises a hand plate and a wrist extension configured for attachment to the arm support. The wrist extension may be slidably or otherwise adjustably and removably coupled to the arm support to provide an arm board device length that may be adjusted to fit a patient's forearm length. In addition to attachment to the arm support, the wrist extension can provide for rotation of the hand plate, whether by a fixed twist or a rotationally moveable coupling. For a right hand plate, the twist can be from about 125 to about 165, or more preferably, about 145 degrees. For a left hand plate, the twist can be from about 15 to about 55, or more preferably, about 35 degrees. The hand plate may comprise an oval shape, circle shape, mitten shape, hand shape, or any other appropriate shape. The hand plate may further comprise a thumb blocker configured to prevent a patient's thumb from reaching the back surface of the hand plate. The hand support can be further configured to allow for limited mobility of the patient's hand to perform various ADL tasks. The hand support can be configured to conform to the posterior, or more preferably, the anterior (palmar) side of the hand. The hand support can comprise an indicator to designate the left and/or right arm and/or hand, for example, a thumb bump-out portion or an “L” or “R” indication. Structural support can be added to the hand support by providing one or more structural support elements (e.g., a C-channel or gusset) along all or a portion of the length of the hand support, for example, along the sides or center. In various embodiments, the hand support is not symmetrical about any longitudinal axis.

The arm support and hand support can comprise a single piece or a plurality of pieces. In various embodiments, the arm support and hand support are distinct pieces that are slidably coupled (e.g., with the dimension defined by the openings between parallel C-channels on one piece corresponding to a dimension of the other piece) to provide adjustability across a wide range of patient sizes, a feature that is useful in imparting a one-size-fits-all benefit. The C-channels may comprise a portion of the structural support described herein; however, other structural support components or configurations are within the scope of the present invention. Once properly adjusted, the slidable overlap between the arm support and hand support can be fixed or substantially fixed by an interference fit, clip(s), clamp(s), bolt(s), screw(s), or the like.

The arm support and hand support can be comprised of one or more suitable materials. Exemplary materials can comprise various plastics, polymers, composites, metals, or the like. Exemplary materials can be smooth to facilitate sterilization after use for re-use. Exemplary materials can be smooth and biocompatible to reduce the risk of skin breakdown in hands, forearms, elbows, or brachium. Exemplary materials can be lightweight and/or waterproof. The arm support and/or the hand support can be manufactured using techniques known in the art, for example, injection molding, casting, etc. An exemplary arm board device may weigh less than about 1000 g, preferably less than about 500 g, and more preferably less than about 300 g. Notwithstanding the foregoing, those skilled in the art will appreciate that the weight may be more or less depending on the materials used. The arm support and hand support can comprise one or more perforations, holes, or the like, to increase breathability. Additional materials may be incorporated to enhance structural support, sterilization, biocompatibility, comfort, etc. For example, medical grade foam and/or wicking materials can be used to cover all or portions of the arm support and/or the hand support. In various embodiments, a removable sleeve covering the arm support and/or the hand support serves a dual function of providing for enhanced sanitization (the sleeve being disposable or sterilizable) and comfort. External padding may also be used around all or a portion of the arm board device to protect the patient and/or medical practitioners. In various embodiments, the arm board device does not comprise an inflatable portion.

In various embodiments, the arm board device may further comprise one or more fasteners or straps (e.g., Velcro fasteners or straps, cam straps, spring buckle straps, ratchet straps, side release buckle straps, strap adjuster straps, double ring straps, and the like) configured to temporarily secure the arm support and/or the hand support to the patient's arm and/or hand. Such fasteners or straps may be connected to the arm board, for example, through a series of loops or the like that are in turn attached to the arm board. In various embodiments wherein such straps comprise Velcro, a portion of the arm support and/or the hand support may comprise a Velcro portion configured to engage one or more Velcro straps. The arm board device may further comprise one or more anchors or attachment points, for example, to connect the arm board device to a hospital bed, if it is desirable to further restrict the patient's mobility.

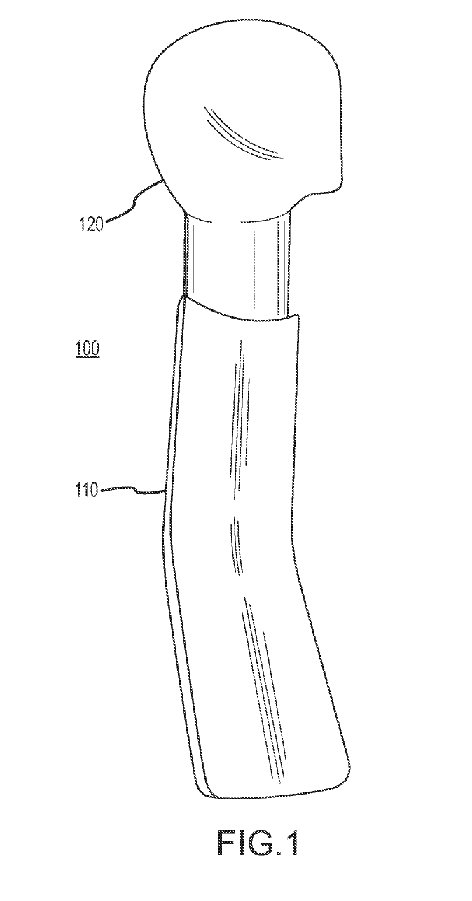

Turning to the Figures, and with reference to FIG. 1, an exemplary arm board device 100 comprises an arm support 110 and, optionally, a hand support 120.

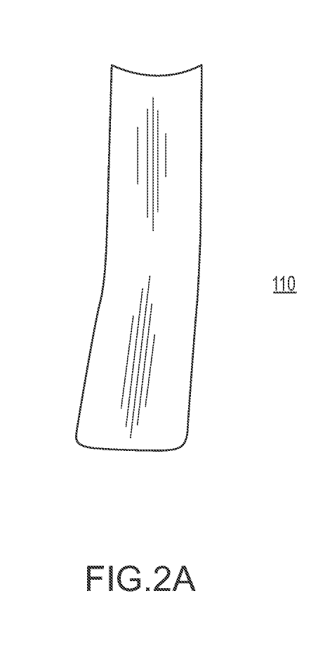

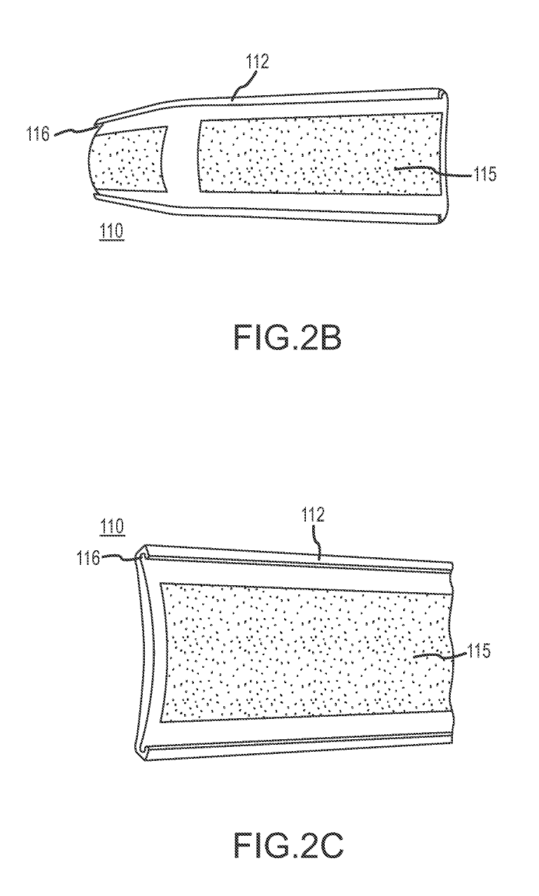

With reference now to FIGS. 2A-2C, an exemplary arm support 110 is 13 inches in length, 3.25 inches wide, and 0.125 inch thick. Arm support 110 is made of hard, shatter and splinter-resistant, smooth plastic FDA-approved material (e.g., acrylonitrile butadiene styrene) having a lateral curvature with a 2 inch radius and a 145 degree angle bend at its center. Arm support 110 comprises two 1 inch strips of Velcro 115 centered along the back surface of arm support 110. Arm support 110 has 13 inch by 0.125 inch arm support C-channels 116 on each longitudinal edge at its back surface.

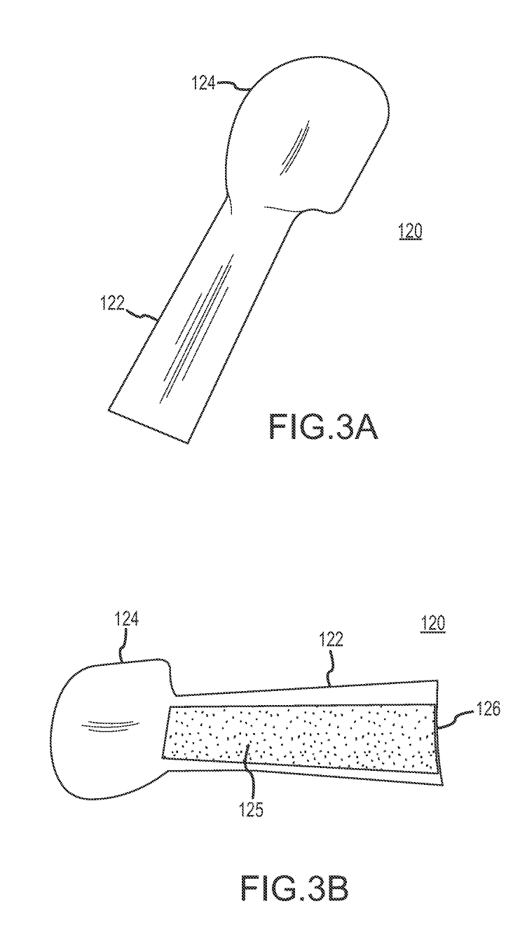

Now with reference to FIGS. 3A-3B, an exemplary hand support 120 comprises a wrist extension 122 and a hand plate 124. The proximal end 126 of wrist extension 122 fits into a dimension defined between the openings of two parallel arm support C-channels 116 (see FIGS. 2B-2C) and is a 10 inch by 3.1875 inch by 0.125 inch piece of acrylonitrile butadiene styrene with a 2 inch radius that tapers down to hand plate 124. At the distal end of wrist extension 122 is a 145 degree fixed twist (for a right hand plate) or a 35 degree fixed twist (for a left hand plate). This rotational bend occurs proximal to hand plate 124 which is a 7 inch by 8.5 inch oval piece of flat acrylonitrile butadiene styrene with a thumb bump out portion on the left or right side of hand plate 124 as appropriate. Hand support 120 comprises a 1 inch strip of Velcro 125 centered along the back surface of hand support 120.

Notwithstanding the foregoing, those skilled in the art will appreciate that the dimensions provided herein vis-à-vis any portion of exemplary arm board devices are scalable up or down, for example, for infants.

In use, the arm board device can be utilized in a two stage process depending upon the needs of the patient. For a patient with maximum safety needs, the arm board device can be placed with the optional hand support, comprising a “stage 1” configuration. The arm board device can easily be placed on the patient's arm and adjusted to make sure that the arm support and the hand support fit comfortably. Straps configured to engage the Velcro strips can be used to secure the arm board device. In a “stage 2” configuration, the optional hand support can be removed. The “stage 2” configuration may be utilized when the patient no longer has medical devices at risk of removal, or other safety needs. Stated differently, the arm board device may be designed to have a dual function. By simply adding or removing the optional hand support, a medical practitioner can quickly change the configuration of the arm board device.

A pilot study to evaluate the effectiveness and safety of the arm board device as an alternative to wrist restraints was conducted with a population of ten patients in an intensive care unit setting. The arm board device was completely effective in preventing removal of any invasive lines or breathing tubes by patients in the treatment group with no incidence of skin breakdown or other adverse effects. Caregiver as well as family evaluations of the device were uniformly positive.

It will be apparent to those skilled in the art that various modifications and variations can be made to the exemplary embodiments of the present disclosure without departing from the spirit or scope of the disclosure. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, materials, elements, components, shape, size and arrangement of parts including combinations within the principles of the invention, to the full extent indicated by the broad, general meaning of the terms in which the appended claims are expressed. To the extent that these various modifications do not depart from the spirit and scope of the appended claims, they are intended to be encompassed therein.

1. An arm board device comprising:
an arm support, wherein the arm support comprises a central portion having an obtuse angle; and
a hand support, wherein the hand support is removably coupled to the arm support,
wherein the hand support comprises a rotational bend of about 145 degrees or about 35 degrees proximal to a distal hand plate of the hand support.

2. The arm board device as in claim 1, wherein the obtuse angle is an angle between about 125 degrees and about 165 degrees.

3. (canceled)

4. The arm board device as in claim 1, wherein the central portion comprises a hinge.

5. The arm board device as in claim 4, wherein the hinge is configured to permit a range of motion of up to 30 degrees.

6. The arm board device as in claim 4, wherein the hinge is configured to have an adjustable range of motion.

7. The arm board device as in claim 1, wherein the arm support is curved orthogonally to a longitudinal axis.

8. The arm board device as in claim 1, wherein the arm support has a structural support element.

9. The arm board device as in claim 8, wherein the hand support further comprises a wrist extension.

10. The arm board device as in claim 9, wherein the wrist extension is slidably coupled with the arm support using a portion of the structural support element.

11. The arm board device as in claim 10, wherein the hand support is further attached to the arm support using Velcro strips.

12. The arm board device as in claim 1, wherein the hand support comprises a rotationally moveable coupling proximal to a distal hand plate of the hand support.

13. (canceled)

14. The arm board device as in as in claim 1, further comprising a Velcro strip.

15. The arm board device as in claim 14, further comprising fasteners configured to secure the arm board device to a patient.

16. The arm board device as in claim 15, wherein the fasteners comprise straps configured to engage the Velcro strip.

17. The arm board device as in claim 1, further comprising anchors configured to attach the arm board device to a bed.

18.-21. (canceled)

22. The arm board device as in claim 1, wherein the arm board device weighs less than 300 grams.

23. (canceled)

24. The arm board device as in claim 1, wherein the arm board device is padded.

25. The arm board device as in claim 1, wherein the arm board device is covered with a removable sleeve.

26. (canceled)

27. The arm board device as in claim 1, wherein a proximal end of the arm support is configured to permit application of a sphygmomanometer cuff to the patient's arm.

28.-39. (canceled)

40. An arm board device comprising an arm support and a hand support,
wherein the arm support comprises a curve orthogonal to a longitudinal axis to provide a configuration suitable for receiving a patient arm;
wherein the arm support comprises about a 145 degree angle at its center;
wherein the arm support comprises a C-channel at a distal end of the arm support;
wherein a proximal end of the hand support is slidably coupled with the C-channel;
wherein the hand support comprises a rotational bend of about 145 or about 35 degrees proximal to a distal hand plate of the hand support;
wherein the arm support comprises a Velcro strip; and
wherein the arm support comprises straps configured to engage the Velcro strip and to secure the arm board device to the patient arm.
